(19) United States
(12) Patent Application Publication
(10) Pub. No.: US 2020/0078455 A1
(43) Pub. Date: Mar. 12, 2020

(54) MESOTHELIN VACCINE FOR OVARIAN CANCER PREVENTION

(71) Applicant: SRI INTERNATIONAL, Menlo Park, CA (US)

(72) Inventors: Nathalie Scholler, Mountain View, CA (US); Paul Stein, Menlo Park, CA (US); Lidia Sambucetti, Redwood City, CA (US)

(21) Appl. No.: 16/607,604

(22) PCT Filed: Apr. 24, 2018
(86) PCT No.: PCT/US2018/029085

§ 371 Date: Oct. 23, 2019

Related U.S. Application Data
(60) Provisional application No. 62/489,238, filed on Apr. 24, 2017.

(51) Int. Cl. A61K 39/00; A61K 45/06; A61P 35/00

(52) U.S. Cl. CPC A61K 39/001168; A61K 45/06; A61P 35/00; A61K 2039/55561; A61K 2039/572; A61K 2039/892; A61K 2039/86; A61K 2039/852; A61K 2039/575; A61K 2039/55566

(57) ABSTRACT

Disclosed are compositions comprising a mesothelin protein and an adjuvant. Disclosed are compositions comprising a mesothelin protein, an adjuvant, and a second adjuvant. In some instances the adjuvant is cyclic dinucleotides (CDNs). Disclosed are compositions comprising a mesothelin protein, CDNs, and a squalene based oil-in-water emulsion. Disclosed are methods of treating cancer comprising administering to a subject a composition or vaccine, wherein the composition or vaccine comprises a mesothelin protein, an adjuvant, and a second adjuvant.

### CROSS-REFERENCE TO RELATED APPLICATIONS

This application claims benefit of U.S. Provisional Application No. 62/489,238, filed Apr. 24, 2017, and is hereby incorporated herein by reference in its entirety.

### STATEMENT REGARDING FEDERALLY SPONSORED RESEARCH

This invention was made with government support under HHSN2612012000141, 2014-5E and 2016-E04 awarded by NCI prevent. The government has certain rights in the invention.

### BACKGROUND

Serous ovarian cancer is the leading cause of gynecological cancer deaths and is mostly detected in late stages while chances of survival at 5 years are lower than 30%. In contrast survival is increased to more than 90% when the cancer is detected at an early stage when it is still localized to the ovaries. Thus, there is an unmet need to develop prevention and early detection strategies for ovarian cancer.

A number of immunodiagnostic and immunotherapeutic approaches against tumors have been developed using mesothelin as a target, including antibody targeting approaches that are currently in clinical trials, and chimeric antigen receptor (CAR) engineered T cells.

### BRIEF SUMMARY

Disclosed are compositions comprising a mesothelin protein and an adjuvant. Disclosed are compositions comprising a mesothelin protein, an adjuvant, and further comprising a second adjuvant. In some instances, the adjuvant is cyclic dinucleotides (CDNs). In some instances, the second adjuvant is a squalene-based-oil-in-water emulsion.

Disclosed are methods of treating cancer comprising administering to a subject a vaccine, wherein the vaccine comprises one or more of the compositions disclosed herein.

Disclosed are methods of triggering an immune response against mesothelin in a subject comprising administering to the subject one or more of the compositions disclosed herein.

Disclosed are methods of immunizing a subject against cancer comprising administering to a subject a vaccine, wherein the vaccine comprises one or more of the compositions disclosed herein.

Disclosed are methods of slowing disease progression in a subject comprising administering to the subject one or more of the compositions disclosed herein.

Disclosed are methods of reducing tumor burden in a subject comprising administering to the subject one or more of the compositions disclosed herein.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a table of the humoral immune response against human and mouse mesothelin proteins after immunization with human mesothelin protein 2.5 ug or 10 ug adjuvanted with Alum/MPL or CDN+/−Addavax™.

FIGS. 2A-2I show the cellular immune response of mice immunized (one prime and 2 boosts) with mesothelin at 2.5 μg (B, C, E-G) or 10 μg (B, D, H-J) in combination with the following adjuvants: CDN (B), Addavax™ (C, D), both (E, F, H, J), or with Alum+MPL (G, J). To control for specificity, mice were immunized with the adjuvants without mesothelin (A). Splenocytes were then assayed for their production of INFγ after incubation with 2 μM of mouse mesothelin or 1 μM of human mesothelin. Splenocytes were incubated with medium only as negative control for proliferation and as positive control with PMA/ionomycine, as indicated.

FIGS. 3A and 3B. 3A shows an experimental design in table form. 3B shows an overview of the experimental schedule.

FIG. 4-Group 1 received Alhydrogel® adjuvant 2 (Aluminum hydroxide gel, “Alum”, 100 mL at 2%) and MPLA Synthetic VacciGrade (TLR4 ligand, “MPL”, 5 μg); group 2 received human mesothelin protein (2.5 μg) combined with Alum and MPL; group 3 received 2′3′-cGAMP VacciGrade (STING ligand, “CDN’, 15 μg) and AddaVax™ (Squalene-Oil-in-water, “Addavax™”, 100 μL); and group 4 received human mesothelin protein (10 μg) combined with CDN 15 μg and Addavax™ 100 μL. FIG. 4 shows human and mouse mesothelin titers 5 weeks after the last immunization and 4 weeks after ID8− Luc injection. ELISA assay were performed with 0.5 μg/mL of human mesothelin and 2 μg/mL of mouse mesothelin. Bound antibodies were detected with anti-mouse total IgG at 1/20,000. Development time was approximately 20 min.

FIGS. 5A-5F show the plots of total flux (p/s) generated by in vivo imaging of the vaccinated mice injected with ID8-Luc mouse ovarian cancer cells. BLI mean of group 1 (A, E) was significantly higher than all the other groups 8 weeks after injection of ID8-Luc ovarian cancer cells. Ten weeks after ID8 Luc injection, BLI means of groups 2 (B) and 3 (C) also increased, but not of group 4 (D). Ten weeks after tumor injection, the mean of BLI signals of group 4 mice was significantly lower than those of all the other groups (ANOVA p=<0.0001) (F).

FIG. 6 shows the three staining panels of antibodies used for flow cytometry. Results were analyzed using FlowJo and Prism to calculate statistical significance per group.

FIGS. 7A-7G show the analysis of lymphocytes in peritoneal lavages. Cells in peritoneal lavages were stained with the antibody panels shown in Tables 1 and 2 and gated on CD45+ CD3− CD19+ for B cells (A) and CD45+ CD3+ for T cells (B). T cells were further gated on CD8+ (C) or CD4+ (D) and characterized for the percentage of CD8 T cells that were (E) IFNγ+ (CTL) or (F) PD-1+ (activated/exhausted), or (G) the percentage of CD4 T cells that were CD25+FoxP3+ (Treg) or CD25−FoxP3− IFNγ− (naïve CD4 T cells), or CD44+ CD62L+ (Memory), or IFNγ+ (ml).

FIGS. 8A and 8B show the analysis of myeloid cells in peritoneal lavages. Cells in peritoneal lavages were stained with the antibody panels shown in the MDCS/MAC/tumor panel (FIG. 6) and gated on CD45+F4_80+ CD11b+ (A) and further gated on (B) PD-L1+ (suppressive myeloid cells), iNOS1+ (proinflammatory macrophages, M1) or MDSC (Gr1+ CD11b+).

FIGS. 9A and 9B show the analysis of tumor cells in peritoneal lavages: Cells in peritoneal lavages were stained with the antibody panel shown in the MDCS/MAC/tumor panel (FIG. 6) and gated on (A) CD45+ EpCAM− or (B) CD45-. Next, CD45− cells were analyzed for EpCAM and/or PD-L1 expression, as indicated.

FIGS. 10A and 10B show the cellular (A) and humoral (B) immune responses of ID8-luc bearing mice immunized with human mesothelin+CDN/Addavax™ against 11 overlapping peptides of 25-mer mapping the sequence of human mesothelin. A: ELISPOT assay. Slenocytes were incubated overnight with 10 μM of human mesothelin peptides or in medium only (unstimulated negative control), or with anti-TCR+anti-CD28 (positive control) as shown, on wells coated with anti-IFNγ antibodies. Signal was detected as recommended by the manufacturer. B. ELISA assay. Sera were incubated in wells coated with 10 μM of human mesothelin peptides or with 1 μg of human mesothelin protein (MSLN, positive control), or non-coated well (0, negative control) as shown. Bound antibodies were detected with HRP-labeled anti-mouse IgG.

FIG. 11 shows an example of an experimental design where mesothelin immunization is administered as a therapeutic vaccine in combination with anti-PD-L1 antibody. FIG. 11 also shows the vaccination of wild type mice 3 months before tumor injection. The immunization protocol is identical to the one described in FIG. 1, except that a group was added that received no immunization.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a mesothelin protein” includes a plurality of such mesothelin proteins, reference to “the mesothelin protein” is a reference to one or more mesothelin proteins and equivalents thereof known to those skilled in the art, and so forth.

As used herein, the term “subject” refers to any organism to which a composition of this invention can be administered, e.g., for experimental, diagnostic, and/or therapeutic purposes. Typical subjects include, but are not limited to, animals, including mammals such as humans and primates; and the like.

As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” cancer can refer to inhibiting tumor growth or metastasis and/or preventing cancer. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range—from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Compositions

Disclosed are compositions comprising a mesothelin protein, or a peptide fragment thereof, and an adjuvant. In some instances, the compositions can be vaccines. In some instances, the mesothelin protein can be 60, 65, 70, 75, 80, 85, 90, 95, or 100% homologous to a full length mesothelin protein. In some instances, the mesothelin protein can be 60, 65, 70, 75, 80, 85, 90, 95, or 100% homologous to a human full length mesothelin protein.

The term mesothelin or mesothelin protein can refer to the 40 kDa glycosylated protein, derived from a 70 kDa precursor that also includes Megakaryocyte Potentiating Factor (MPF). The 70 kDa precursor is cleaved at a dibasic proteolytic site to release the 32 kDa glycosylated MPF. Alternate splicing can generate mesothelin isoforms that have either an eight amino acid insertion following Ser408 or a substituted C terminal region with no GPI anchor.

In an aspect, recombinant human mesothelin protein can be used. In an aspect, a recombinant human mesothelin protein, CF can be purchased from R&D systems, catalogue number 3265-MS-050. This recombinant human mesothelin lacks the 8 aa insertion, and within aa 296-580 it shares 59% sequence identity with mouse and rat mesothelin. The recombinant mesothelin consists of residues Glu296-Gly580, with a C-terminal 6-His tag and is produced in NS0, a heterologous mammalian expression system that allows for the rapid expression of recombinant proteins.

In some instances, the adjuvant can be capable of binding to the stimulator of interferon genes (STING). In some instances, the adjuvant can be cyclic dinucleotides (CDNs). In some instances, CDNs can be synthetic. CDNs can be, for example, 2′3′-cGAMP, 5,6-Dimethylxanthenone-4-acetic acid (DMXAA), IC31, dithio-(R_{P}, R_{P})-[cyclic[A(2′,5′)pA(3′,5′)p]], (ML RR-S2 CDA).

In some instances, the mesothelin protein can be recombinant. In some instances, the mesothelin protein can be a naturally occurring purified mesothelin protein.

In some instances, the mesothelin protein can be the full length human mesothelin protein. (MSLN1 NP_005814.2, MSLN2 NP_037536, MSLN Variant 3 ref|NM_001177355.1|.

AAH09272.1 (aa 296-580). In some instances, the mesothelin protein can be the full length mesothelin protein of another mammalian species, such as, but not limited to, chicken mesothelin (XM_414835). For example, chicken mesothelin can be used for vaccination of battery-farmed chicken against ovarian cancer.

In an aspect, disclosed is a composition that comprises a recombinant human mesothelin protein produced by R&D Systems combined with synthetic CDN 2′3′-cGAMP (mlCDN, 2′3′-cGAMP VacciGrade™, Invivogen). In an aspect, disclosed is a composition that comprises a recombinant human mesothelin protein produced by R&D Systems combined with synthetic CDN 2′3′-cGAMP (mlCDN, 2′3′-cGAMP VacciGrade™, Invivogen) and Addavax™ (AddaVax™, 50 μl).

The disclosed compositions can further comprise a second adjuvant. In some instances, the second adjuvant can be a squalene-based-oil-in-water emulsion. For example, a squalene-based-oil-in-water emulsion can be AddaVax™.

In an aspect, disclosed is a composition that consists of a vaccine that is composed of recombinant human mesothelin protein produced by R&D Systems (described below) combined with synthetic CDN 2′3′-cGAMP (mlCDN, 2′3′-cGAMP VacciGrade™, Invivogen) plus Addavax™ (AddaVax™, 50 μl). AddaVax™ is a squalene-based oil-in-water nano-emulsion with a formulation similar to MF59® that has been licensed in Europe for adjuvanted flu vaccines.

In some instances, the disclosed compositions can further comprise an immunomodulatory agent. In some instances, the immunomodulatory agent can enhance the immune response. For example, checkpoint blockades such as the immunomodulatory agent can inhibit PD-1, anti PD-L1, or CTLA-4.

Disclosed are compositions comprising a mesothelin protein, CDN, and Addavax™ In some instances, disclosed are compositions comprising 10 μg of mesothelin protein plus 15 μg of CDN with 100 μL of Addavax™. In some instances, the composition can comprise 5, 10, 15, 20, 25, 30, 35, 40, 45, or 50 μg of mesothelin protein. In some instances, the composition can comprise 10, 15, 20, 25, 30, 35, 40, 45, 50, 55, or 60 μg of CDN. In some instances, a composition comprising 10 μg of mesothelin protein plus 15 μg of CDN with 100 pt of Addavax™ can be used for immunizing/treating small animal, such as mice. Thus, these concentrations can be scaled up for larger animals, such as humans. In some instances, the Addavax™ can be replaced with any adjuvant. For example, any squalene-based-oil-in-water emulsion can be used.

1. Vaccines

As described herein, mesothelin proteins can be used in a vaccine. For example, disclosed herein are vaccines which decreases the number of boosters required to obtain memory cells comprising a mesothelin protein, or a fragment thereof and a pharmaceutically acceptable excipient. Also disclosed are vaccines which decreases the number of boosters required to obtain memory cells comprising a mesothelin protein, or a fragment thereof and a pharmaceutically acceptable excipient, further comprising a suitable adjuvant.

Also disclosed are vaccines which decrease the amount of time for full memory cell response, comprising a mesothelin protein, or a fragment thereof and a pharmaceutically acceptable excipient. Also disclosed are vaccines which decrease the amount of time for full memory cell response, comprising a mesothelin protein, or a fragment thereof and a pharmaceutically acceptable excipient, further comprising a suitable adjuvant.

The establishment of immunological memory is one of the goals of vaccine development. Yet, the establishment of immunological memory can take months to occur following the initial antigenic encounter. Additionally, the mere establishment of immunological memory is not necessarily sufficient to confer protection against future encounters with a pathogen or foreign antigen, as a small memory population may be overwhelmed by a pathogen. Therefore an additional goal is to establish a memory population large enough to provide the protection. For vaccine development, the sufficiency of the immunological memory can be improved through the administration of additional applications of the same or related antigens as the initial vaccine, referred to as a boost. However, multiple boosts may be required and current immunization regimens often require months between successive vaccine administrations. Thus, a continued problem plaguing vaccine development is the establishment of an effective means to rapidly establish protective immunity.

The establishment of a long-lived immune response to a target that is of a size large enough to protect the recipient and generated quickly enough to meet the needs of those receiving a vaccine is the continuing goal in the development of many vaccines. Vaccines refer to any composition that is administered to a subject with the goal of establishing an immune response to a particular target or targets. In certain embodiments the vaccines will produce an immune response that is a protective immune response. Vaccines can be, for example, prophylactic, that is, administered before a target is ever encountered, as is typically the case for Polio, measles, mumps, rubella, smallpox, chicken pox, and influenza vaccines, for example. Vaccines can also be therapeutic, providing an immune response to a target that is already within a subject, for example, a vaccine to a particular cancer. Typically vaccines are administered in a single or multiple doses called immunizations and are designed to generate memory T and B-cell populations. However, to date, no vaccine designed to generate memory T-cells has accomplished this task with a single dose, or immunization, of the vaccine. Often with vaccines directed to T-cell immunity, the initial immunization, or prime, generates a memory T-cell population that is insufficient to provide protection against future target encounter related to the antigen. Additionally, the few memory T-cells that are generated from the initial prime can take at least 2 months and can take years to finally transform from naïve T-cells into memory T-cells. To overcome the problem of inadequate initial priming, additional immunizations, or boosts, comprising the same or related antigen are used to bolster the numbers of memory T-cells. However, for a boost to be effective, the memory T-cell population must be stabilized. That is, the target-specific T-cell population must have completed the transformation to memory cells and be in a steady-state. Thus, a prime-boost immunization regimen can require months between immunizations creating a tremendous lag in time between when immunity to a target is desired and when it is actually achieved. The methods disclosed herein overcome these problems.

Typically, memory T-cells can be characterized as long-lived antigen-specific T-cells having a combination of two or more of the following markers CD44⁺ (positive), CD11a⁺ (positive), CD43^{1B11-} (negative), CD62L^{HI or LO} CD127⁺ (positive), and CD45RA⁻ (negative), CD27^{hi}, CD122^{hi}, IL-15R+. Memory T-cells can be divided into two major groups distinguished by the expression of CCR7 and CD62L. CCR7⁻, CD62L^{lo} (negative) memory T-cells are referred to as “effector memory T-cells” (T_{EM}). These cells generally are localized in the peripheral tissues such as the liver and lungs as well as the spleen, and produce rapid effector functions, such as IFN-α production, upon stimulation. CCR7⁺ (positive) memory T-cells generally localize in the secondary lymphoid organs such as the thymus, bone marrow, and lymph nodes, although they can also be found in peripheral tissues. These cells are referred to as “central memory T-cells” (T_{CM}) and provide more effective protection to the host, against at least some pathogens, through increased proliferative capacity. It is understood that maintained within a population of memory T-cells is the potential for further expansion upon future antigen encounter. Thus, herein disclosed are methods of generating memory T-cells. The memory T-cells can be generated, for example, by mixing a target or antigen related to the target with dendritic cells and administering the mixture to a subject. It is understood that the disclosed methods can be used for the generation of, for example, central memory T-cells.

It is also contemplated that the booster immunization can comprise any antigen related to the target including, but not limited to, the same antigen supplied in the mixture provided in the prime comprising an antigen related to the target and a dendritic cell. Thus, it is understood that the antigen provided in the booster can be different from the antigen in the prime. It is also understood that the antigen provided in the booster can be different than mesothelin. It is further understood that the disclosed methods can comprise more than one boost.

2. Compositions, Characteristics, and Relationships

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular mesothelin protein is disclosed and discussed and a number of modifications that can be made to a number of molecules including the mesothelin are discussed, specifically contemplated is each and every combination and permutation of mesothelin and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

i. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

ii. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

iii. Nucleic acids

There are a variety of molecules disclosed herein that are nucleic acid based, including for example the nucleic acids that encode, for example, human mesothelin (MSLN1 NP_005814.2, MSLN2 NP_037536, MSLN Variant 3 ref|NM_001177355.1|) as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

iv. Peptides and Proteins

a. Protein Variants

As discussed herein there are numerous variants of the mesothelin protein that are known and herein contemplated. In addition, to the known functional strain variants there are derivatives of mesothelin proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic | Glu |
| acidp | |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative
Substitutions, others are known in the art.

ala; ser
arg; lys, gln
asn; gln; his
asp; glu
cys; ser
gln; asn, lys
glu; asp
gly; pro
his; asn; gln
ile; leu; val
leu; ile; val
lys; arg; gln;
met; leu; ile
phe; met; leu; tyr
ser; thr
thr; ser
trp; tyr
tyr; trp; phe
val; ile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T.E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, the sequences of MSLN1 NP_005814.2, MSLN2 NP_037536, MSLN Variant 3 ref|NM_001177355.1 set forth a particular sequence of human mesothelin protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in MSLN1 NP_005814.2, MSLN2 NP_037536, MSLN Variant 3 ref NM_001177355.1. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

v. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

vi. Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

3. Therapeutic Uses

Effective dosages and schedules for administering the compositions may be determined empirically, and making such determinations is within the skill in the art. The dosage ranges for the administration of the compositions are those large enough to produce the desired effect in which the symptoms and disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Methods of Treating

Disclosed are methods of treating cancer comprising administering to a subject a vaccine, wherein the vaccine comprises one or more of the compositions disclosed herein. The vaccine can be prophylactic or therapeutic.

In some instances, the cancer can be any cancer in which mesothelin is overexpressed, such as, but not limited to, ovarian, lung, pancreatic cancer, and brain cancers such as Leptomeninges and Meningiomas.

The disclosed methods of treating cancer can further comprise administering an immunomodulatory agent. In some instances, the immunomodulatory agent enhances the immune response. For example, checkpoint blockades with immunomodulatory agent that can inhibit PD-1, anti PD-L1, or CTLA-4.

In some instances, the immunomodulatory agent can be administered simultaneously with the vaccine. In some instances, the immunomodulatory agent can be a part of the vaccine, thus the vaccine and immunomodulatory agent are administered together. In some instances, the immunomodulatory agent is separate from the vaccine. In some instances, the immunomodulatory agent can be administered within hours, days, or weeks of the vaccine.

In some instances, the disclosed methods of treating cancer further comprise detecting an overexpression of mesothelin in the ovaries of the subject prior to administering the vaccine. The detection of the overexpression of mesothelin in the ovaries confirms that the subject is in need of the disclosed vaccine.

### D. Methods of Triggering Immune Response

Disclosed are methods of triggering an immune response against mesothelin in a subject comprising administering to the subject one or more of the compositions disclosed herein.

In some instances, the immune response that is triggered can be a Th1 immune response. In some instances, the immune response that is triggered can be a Th2 immune response. In some instances, the immune response that is triggered can be both a Th1 and Th2 immune response.

### E. Methods of Immunizing

Disclosed are methods of immunizing a subject against cancer comprising administering to a subject a vaccine, wherein the vaccine comprises one or more of the compositions disclosed herein.

In some instances, the cancer can be can be any cancer in which mesothelin is overexpressed, such as, the cancer can be, but is not limited to, ovarian, lung or pancreatic cancer, also certain subtypes of brain cancer such as Leptomeninges and Meningiomas.

In some instances of the disclosed methods of immunizing, mesothelin specific antibodies can be increased in the subject. In some instances, mesothelin-specific cytotoxic CD8+ T cells are elevated. Thus, a Th1 response, Th2 response, or both can be activated upon immunization.

In some instances, the subject has previously been determined to be at risk for developing cancer. In some instances, the subject has previously been diagnosed with cancer.

### F. Methods of Slowing Disease Progression

Disclosed are methods of slowing disease progression in a subject comprising administering to the subject one or more of the compositions disclosed herein. The slowing of disease progression can be related to any disease wherein mesothelin is overexpressed. For example, the disease can be cancer, such as, but not limited to, ovarian, lung or pancreatic cancer.

### G. Methods of Reducing Tumor Burden

Disclosed are methods of reducing tumor burden in a subject comprising administering to the subject one or more of the compositions disclosed herein.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for producing a vaccine, the kit comprising a mesothelin protein and an adjuvant. The kits also can contain a second adjuvant.

In some instances, the adjuvant is CDN. In some instances, the second adjuvant is Addavax™.

### EXAMPLES

### A. Overview

The approach to prevent ovarian and other mesothelin expressing cancers consists of generating protective immunity using a protein-based vaccine consisting of mesothelin and a specific adjuvant that binds to the stimulator of interferon genes (STING) and promotes cell-based immunity called, cyclic di-nucleotides (CDNs). It was anticipated that CDNs would activate the interferon (IFN) pathway thus adding CDNs to the vaccine could result in elevated antigen-specific cytotoxic CD8+ T cells and other tumor-targeting immune cells that are capable of infiltrating and destroying the tumor. The FDA has approved only a few adjuvants and the most common one, alum, elicits only a poor cell-mediated immune response and a Th2-biased immune response, which is not optimal as a cancer vaccine. In fact, most protein-based vaccines using currently available adjuvants fail to promote a robust CD8+ T cell response, limiting their potential effectiveness. For example, the combination of alum and MPL, a TLR4 agonist, has been FDA-approved but failed to mount a broad-based CD8+ T cell response. Other TLR ligands, such as CpG, are in development because they elicit a strong Th1-biased immune response, but reports of CpG-mediated generation of CD8+ T cell responses have been inconsistent. The combined administration of mesothelin with an adjuvant that promotes the activity of cytotoxic CD8+ T cells is a novel efficacious vaccine approach to prevent cancer.

Mesothelin is an immunogenic protein that is overexpressed in ovarian, pancreatic, lung cancers, and brain cancers such as Leptomeninges and Meningiomas, such that the vaccine may be effective against all these cancer types. While healthy women in general can benefit from a vaccine to prevent ovarian cancer, women in higher risk categories may be the most strongly indicated to receive the vaccine. There is a distinct hereditary risk associated with specific subpopulations of ovarian cancer. While BRCA1/2 mutations are strongly associated with breast cancer, these mutations also confer a 20-40% risk towards developing ovarian cancer as well. Similarly, women with hereditary non-polyposis colorectal cancer also have a 10% elevated risk of developing ovarian cancer. These groups would be prime candidates for receiving a preventative ovarian cancer vaccine.

Men and women at risk for lung or pancreatic cancer can also benefit from protection from this vaccine. Additionally, the vaccine may confer therapeutic benefit by helping to prevent ovarian cancer recurrence in women in following treatment with chemotherapy. Commercial impact may be very significant. For example, the market for the HPV vaccine for prevention of cervical cancer is forecast to reach US$2.2 billion in 2018, with GSK and Merck competing for this market.

Although research has shown that ovarian cancer vaccines may hold promise, these studies have involved only small numbers of participants and have focused on prevention of recurrance following chemotherapy. Researchers are studying how cancer vaccines are best used in combination with chemotherapy and other treatments. Some ovarian cancer vaccines that have been studied include: Abagovomab and Oregovomab. Abagovomab has been shown to elicit an immune response in women with ovarian cancer, but it's not clear that this leads to longer survival. Oregovomab also has been tested in women and has been shown to elicit an immune response. But one study showed no difference in the recurrence rate in women who got oregovomab as compared with women who received a placebo.

The current approach is distinguished by a potential to prevent disease and a focus on mobilizing cell-based immune defenses. Further, the animal studies indicate that vaccinated animals are protected from disease progression when challenged with ovarian cancer.

The current objective is to develop a protein-based vaccine that triggers both humoral and cellular immune responses against tumor antigen(s) such as mesothelin that is overexpressed by tumor cells. The use of adjuvants can modulate the intensity and the quality of the immune response. Immunization of wild type mice with mesothelin in combination with cyclic dinucleotides (CDNs), a relatively new class of adjuvants that activate innate immunity and/or squalene-based oil-in-water nanoemulsions that recruit APC, promotes an anti-mesothelin immune response with high levels of mesothelin-specific antibodies and elevated antigen-specific cytotoxic CD8+ T cells capable of infiltrating and destroying mesothelin-expressing ovarian cancer.

Disclosed herein are studies using a vaccine that is composed of recombinant human mesothelin protein produced by R&D Systems (described below) combined with synthetic CDN 2′3′-cGAMP (mlCDN, 2′3′-cGAMP VacciGrade™, Invivogen) plus Addavax™ (AddaVax™, 50 μL). AddaVax™ is a squalene-based oil-in-water nano-emulsion with a formulation similar to MF59® that has been licensed in Europe for adjuvanted flu vaccines. All the individual components were acquired commercially.

The components of the vaccine described above were optimized using a murine model of ovarian cancer. Amounts of mesothelin and adjuvants were titrated and the vaccine efficacy was compared to other clinically approved adjuvants. Immunization with 10 μg of mesothelin plus 15 μg of CDN and Addavax™ was the most efficient combination to mount a Th1 polarized immune response against mesothelin, and thus was most likely to protect mice against mesothelin expressing cancer cells. The vaccine was tested in mice orthotopically implanted with mouse ovarian cancer cells and found that the vaccine protected the mice, significantly reducing the tumor burden at early time points, prolonging the time to disease progression and reducing the percent of mice that developed ascites. Furthermore, the mesothelin+CDN/AddaVax™ vaccinated mice were shown to have increased markers of cell-mediated immunity.

These results were surprising and could not have been anticipated, since other clinically approved adjuvant combinations were also able to elicit strong humoral immune responses; however, these other adjuvant combinations did not protect the animals from ovarian cancer and did not produce the protective cellular immune response.

As adjuvant control, a combination of Alhydrogel® adjuvant (Alum, 50 μL) was used in gel suspension at 2% that optimizes the activation of NLRP3 inflammasome complex and improves antigen attraction and uptake by APCs, plus MPLA Synthetic VacciGrade™ (MPL, 5 μg), a synthetic lipid A from E. coli serotype R515 that specifically activates TLR4 and was reported to induce a strong Th1 response in mice. Adding these adjuvants (Alum plus MPL) to the mesothelin vaccine, in place of CDN 2′3′-cGAMP plus Addavax™ produced high and titers against both human and mouse mesothelin in mice immunized with 2.5 μg of mesothelin. Despite a robust humoral immune response, mice vaccinated with mesothelin and Alum plus MPL were not protected from development of orthoptopically implanted ovarian cancer.

### B. Mesothelin Vaccination for the Prevention of Ovarian Cancer—Part I

1. Introduction

The current objective is to develop a protein-based vaccine that triggers both humoral and cellular immune responses against mesothelin that is overexpressed by ovarian cancer cells. The use of adjuvants can modulate the intensity and the quality of the immune response. Immunization with mesothelin in combination with cyclic dinucleotides (CDNs), a relatively new class of adjuvants that activate innate immunity and/or squalene-based oil-in-water nano-emulsions that recruit APC, can promote an antitumor immune response with high levels of mesothelin-specific antibodies and elevated antigen-specific cytotoxic CD8+ T cells capable of infiltrating and destroying mesothelin-expressing ovarian cancer.

Various combinations and amounts of adjuvants, including CDNs, were compared for their ability to stimulate an immune response to human mesothelin in wild type C57Bl/6 mice. The results of the IFN gamma ELISPOT remained of a concern at the time of the last progress report, thus it was only tentatively concluded that immunization with 10 μg of mesothelin in CDN 15 μg+Addavax™ (100 uL) was the most efficient to mount a Th1 polarized immune response against mesothelin, and thus the most likely to protect mice against mesothelin expressing cancer cells. Current results confirm this.

2. Technical Activities, Status and Results

i. Summary of Ex Vivo Analysis of the Immune Responses of WT Mice Immunized with Mesothelin Combined with Various Adjuvants.

a. Humoral Immune Response

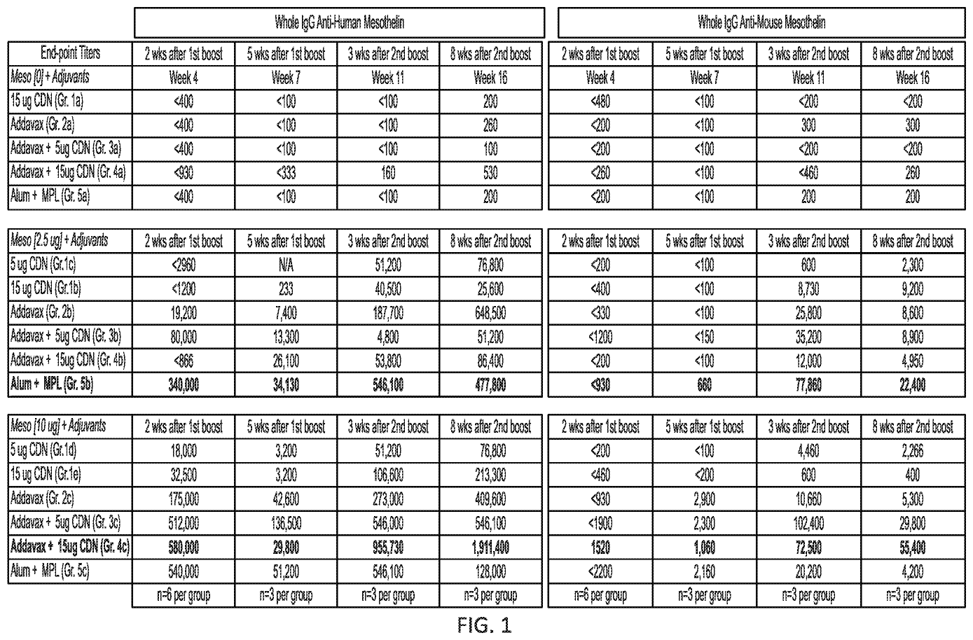

The ELISA assays showed that the titers of antibodies against human mesothelin were stable from week 3 to week 9 or even increased in some cases, and the titers of antibodies against mouse mesothelin only slightly declined from week 3 to week 9 after the 2nd boost (FIG. 1). These results favorably compare to the IgG titers after the 1st boost that sharply declined between week 4 and 7. In addition, the highest and most stable titers against both human and mouse mesothelin were observed in mice immunized with 10 μg of mesothelin in combination with Addavax™ and 15 μg of CDN (Group 4c). The second best titers were found in mice immunized with 2.5 μg of mesothelin in combination with Alum and MPL (Group 5b).

b. Cellular immune response

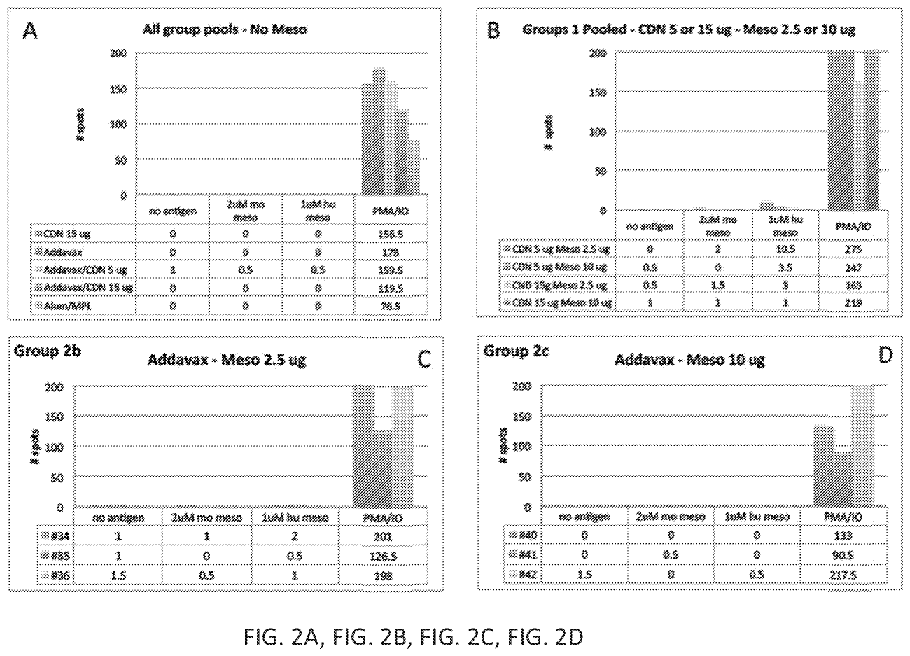

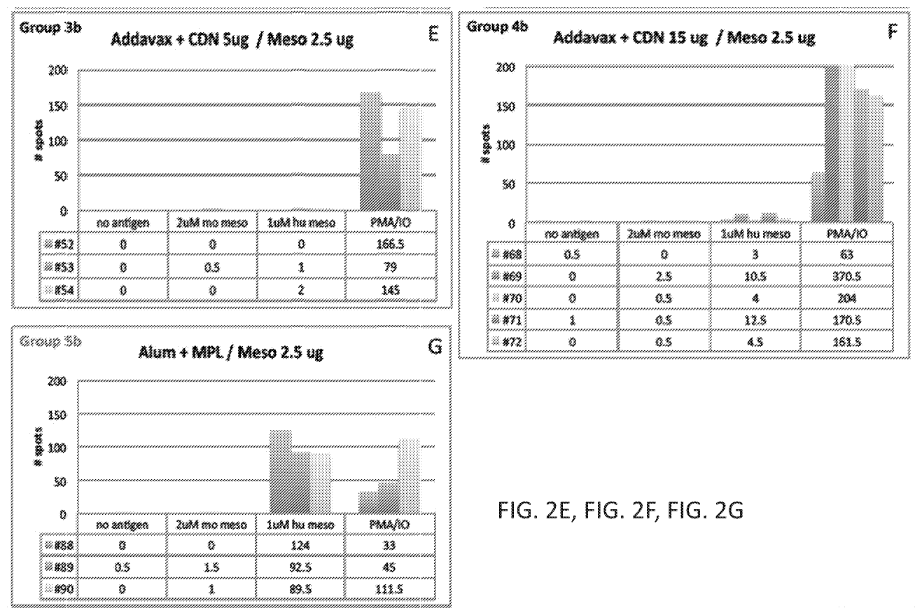

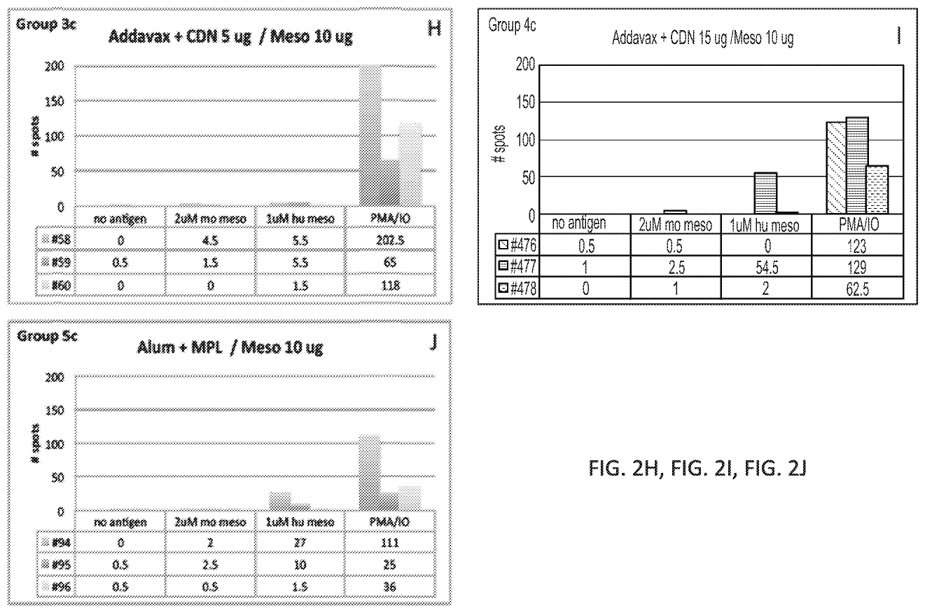

Next, the IFNγ ELISpot assays showed production of IFNγ in response to human mesothelin by the splenocytes of all tested mice from Group 5b (FIG. 2). None of the immunized mice showed production of IFNγ in response to stimulation with mouse mesothelin.

As shown in FIG. 1, while the IgG end-point titers against mouse mesothelin are high (about 50,000), there are still at least one log lower than the IgG end-point titers against human mesothelin.

These results combined with other results, and in particular the ratio IgG2b/IgG1 showing that immunization of the group 4c was the most efficient in polarizing the immune response toward Th1/cellular immunity, strongly indicated that the immunization protocols conducted in groups 4c and 5b are the most likely to protect the mice against mesothelin-expressing cancers, such as ovarian cancer induced by the orthotopic injection of a mouse ovarian cancer cell line.

ii. Immunization of WT Mice Bearing Orthotopic Ovarian Cancer Cells with Mesothelin Combined with Alum/MPL or CDN/Addavax™.

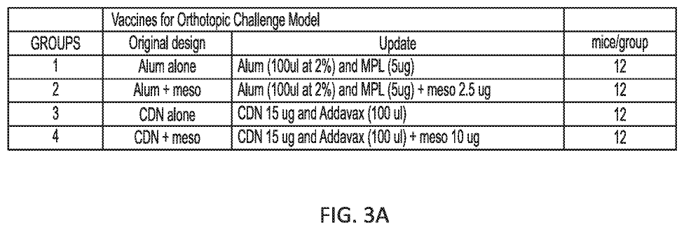

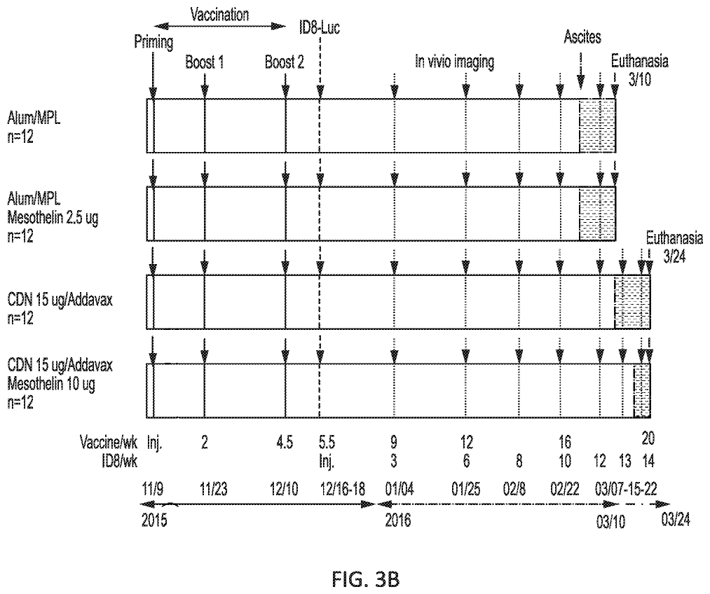

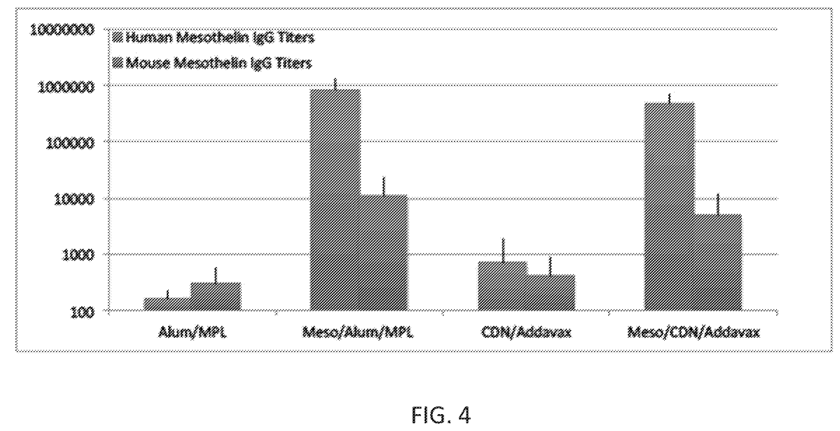

Luciferase-transduced ID8 cell line (Luc-ID8) were injected orthotopically in mice immunized with Alum/MPL+/−2.5 μg of mesothelin, or CDN 15 μg/Addavax™+/−10 μg of mesothelin as described in FIG. 3A. The details of the experimental schedule are summarized in FIG. 3B. As diagramed in FIGS. 3A and 3B, mice were first immunized on November 9, then boosted on November 23 and December 11, and finally injected intraovary with ID8 mouse cancer cells on December 16-18. The first in vivo imaging to visualize the presence of tumor cells was conducted the first week of January 2016.

Because in the ID8 model, mice consistently develop ascites 12 to 14 weeks after tumor injection, the groups not receiving mesothelin recombinant protein were anticipated to develop ascites, while the ascites development would be delayed or stopped in the 2 other groups. Mice were euthanized about 14 weeks after tumor injection, and analyzed for their immune response and tumor burden.

### C. Mesothelin Vaccination for the Prevention of Ovarian Cancer

1. Introduction

Most patients with high-grade serous ovarian cancer (HGSC) are diagnosed with late stage, metastatic disease, when survival rates remain poor and the relapse rates are high despite recent advances in surgical and pharmaceutical therapies. However, when ovarian cancer treatment can be administrated during the early stage of the disease, when the disease is still localized to the ovary, patient survival at 5 years is usually about 90%. The current objective is to develop a protein-based vaccine that triggers both humoral and cellular immune responses against mesothelin that is overexpressed by ovarian cancer cells, and that can protect against or delay metastatic disease. The use of adjuvants can modulate the intensity and the quality of the immune response. Immunization with mesothelin in combination with cyclic dinucleotides (CDNs), a relatively new class of adjuvants that activate innate immunity and/or squalene-based oil-in-water nano-emulsions that recruit antigen-presenting cells (APC), would promote an anti-tumor immune response with high levels of mesothelin-specific antibodies and elevated antigen-specific cytotoxic CD8+ T cells capable of infiltrating and destroying mesothelin-expressing ovarian cancer. The best combinations and amounts of adjuvants were identified for their ability to stimulate an immune response to human mesothelin in wild type C57Bl/6 mice and immunization with 10 μg of mesothelin in CDN 15 μg+Addavax™ was the most efficient to mount a Th1 polarized immune response against mesothelin. Next immunized mice were injected intra ovary with ID8-luc, an ovarian cell line transduced with luciferase, and tumor growth and immune response were monitored. The results support the hypothesis that a combination of mesothelin protein in combination with CDN and Addavax™ can trigger both humoral and cellular immune responses against mesothelin and significantly delay the development of metastatic ovarian cancer.

2. Technical Activities, Status and Results

i. Short Term Immunization with ID8-Luc

a. Experimental Design

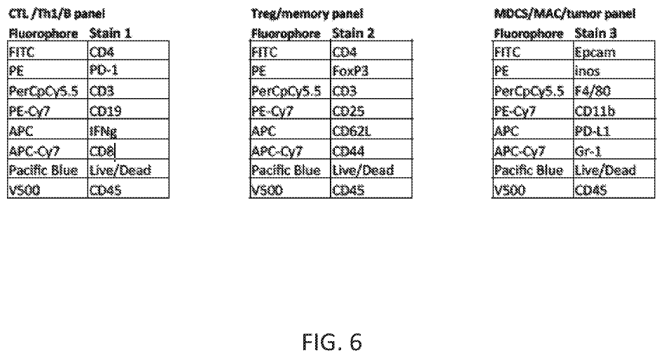

Four groups of 12 C57BL/6 mice were immunized with mesothelin protein and 2 different adjuvant formulations, as shown in FIG. 6. All groups were immunized with one primary injection followed by 2 boosts delivered approximately 2 weeks apart. One week after the end of the vaccination regimen, five million of luciferase expressing ID8 ovarian carcinoma cells (ID8-Luc) were orthotopically injected into the ovarian bursa of all the vaccinated mice. Of note, in group 4 one animal died 4 weeks after ID8-Luc injection and one animal had to be euthanized 6 weeks after ID8 injection due to infected skin rash.

b. Humoral Immune Response of Immunized Mice.

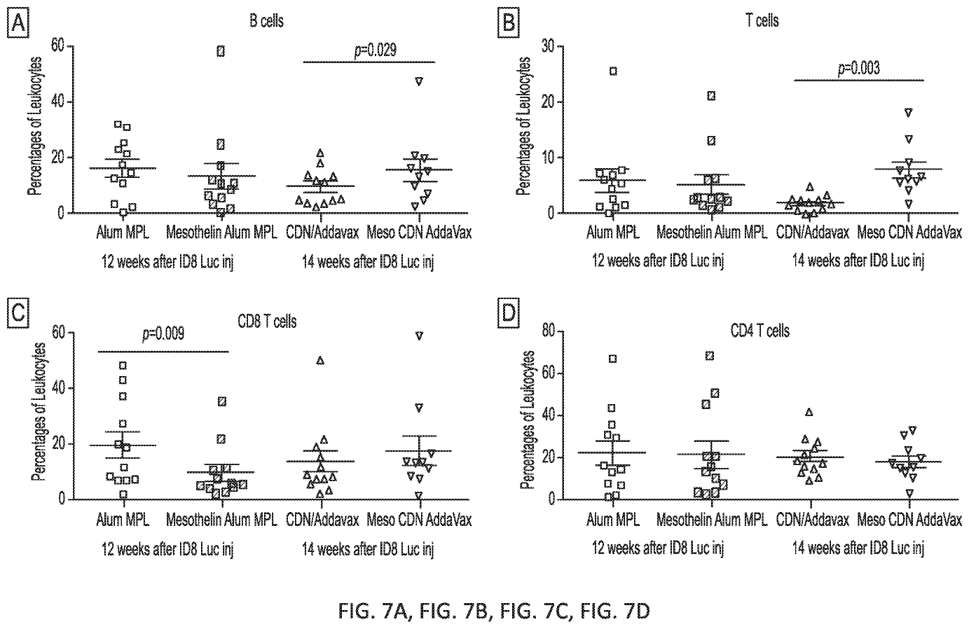

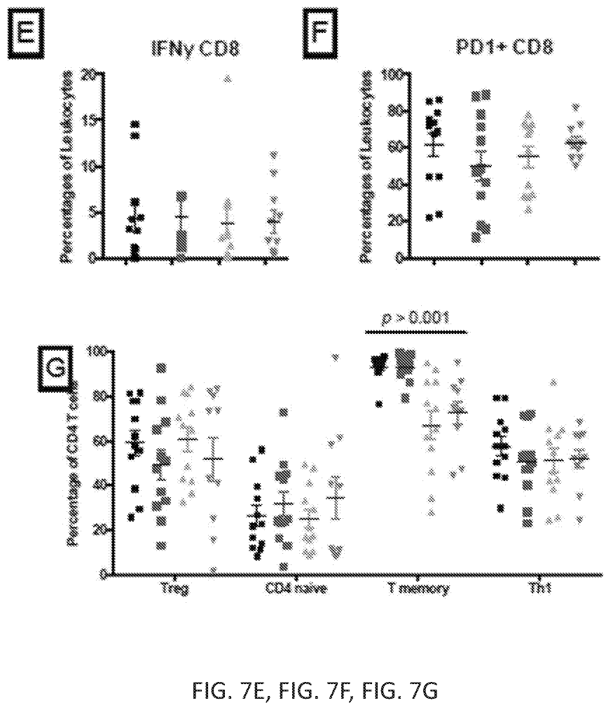

ELISA assay to measure mesothelin antibodies in peripheral sera was performed 10 weeks after the beginning of the immunization that is 4 weeks after ID8 injection. IgG titers against human and mouse mesothelin were determined as described in the previous progress reports and showed that the mice immunized against human mesothelin mounted a specific humoral immune response against both human and mouse mesothelin (FIG. 7). Of note, mice immunized with CDN/Addavax™ and to a lesser extent with Alu/MPL, appeared to have a low level of anti-mesothelin antibodies, suggesting that this immune response might have been stimulated by the adjuvants in response to mesothelin produced by the ID8 tumors.

c. Tumor Growth Monitoring

ID8-Luc cells express the firefly reporter enzyme that generates a photon flux (light) when luciferin (the luciferase substrate) is oxidized in the presence of ATP. As a consequence, only live tumor cells that express the enzyme are detected, rendering bioluminescent imaging (BLI) an excellent and sensitive tool to examine tumor growth in mouse models. Light is absorbed, scattered, and refracted as it traverses tissues and blood and the greater the distance the light travels through tissue before exiting the skin, the greater the attenuation of the signal. Longer wavelengths (especially in the infrared) pass through tissue with less attenuation, but in the range of peak absorbance for firefly luciferase (near 500 nm), there is significant tissue scattering and absorbance of light. Therefore, in the case of ID8-luc-induced ovarian cancer, BLI is an accurate tool during the early development of the disease. But when fluid accumulates in the abdominal cavity, the light passing through the abdomen can be attenuated thus decreasing the photon flux that can be measured. Hence the measurements are not proportional to tumor growth once ascites develops.

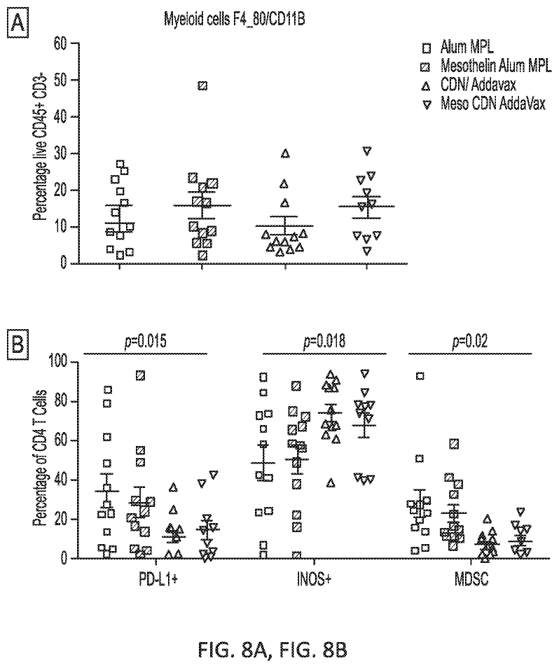

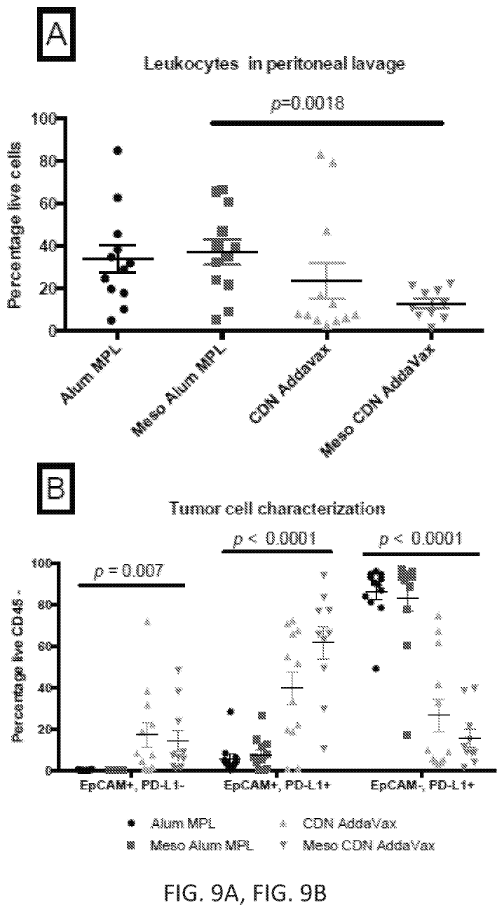

In vivo imaging was performed 3, 6, 8, 10, 12, 13 and 14 weeks after the injection of ID8-luc (FIG. 8). Several animals of group 1 showed sudden and large increase of bioluminescent signal as early as 8 weeks after tumor injection. Ten weeks after the injection, the bioluminescent signals of about half of the animals of groups 1, 2 and 3 were significantly increased (FIG. 8A-C). In sharp contrast, BLI signals of group 4 were still at background level (FIG. 8D, E). After ten weeks, the likely presence of fluid in the abdominal cavity of some animals attenuated the light passing through the abdomen, which translated into the decrease of the BLI signals that did not correlate with the tumor burden but likely correlated with development of ascites. Notably, in groups 1, 2 and 3 a few animals had a sharp rise in BLI signals at weeks 10-12 that were followed by a sharp decline (FIG. 8A-C). Animals of groups 1 and 2 were sacrificed at week 12, as well as one animal of group 3 that had an ascites (#3.46). Metastatic disease was found in 12/12 animals of group 1 and 11/12 animal of group 2. In addition, 2/3 of the animals in these groups had large bloody ascites. In group 2, unusual features were observed during the dissection in addition of a large tumor burden: animal #2.33 presented a large inflammatory area between the skin and peritoneum; #2.41 has a large cyst on the injection site; #2.22 and #2.36 presented red and swollen fallopian tubes; finally one animal (#2.43) had a smaller tumor burden (FIG. 9B). Two other animals of group 3 presented an ascites at week 13 and needed to be sacrificed (#3.2 FIG. 9A, #3.18).

At week 14, 3 animals of group 4 suddenly developed larger tumor burden (animals #4.31, #4.9 and #4.29). In vivo imaging revealed one animal (4#9, FIG. 8D, FIG. 9A) and clinical examination identified two other (#4.31 and #4.29) showing again the limit of BLI for advanced disease (FIG. 9). The experiment was terminated at week 14. Autopsy showed that 6 out of 9 remaining animals of group 3 presented metastatic disease and 3 had bloody ascites (#3.6, #3.15, #3.16). Three animals had unilateral tumors only (#3.3, #3.10 and #3.25). In summary for group 3, 9 out 12 animals developed a metastatic disease from week 12 to 14 after tumor injection. In contrast, all animals of group 4 looked healthy until week 13th after tumor injection, and only 3/10 animals of group 4 (#4.31, #4.9 and #4.29) presented bloody ascites with metastatic disease at week 14. Six animals out of 10 only presented small unilateral tumors and the mice were generally healthy, exhibiting normal behavior, groomed fur and abdominal fat (#4.8 and #4.26 shown in FIG. 9B, #4.13, #4.24, #4.34 and #4.49).

At the time of sacrifice, blood was harvest for serum storage, spleens, peritoneal lavages (in 5 mL of PBS in the absence of ascites), ovaries and fallopian tubes for all groups, and bone marrows for groups 3 and 4 only. Spleens and bone marrows were dispersed in single cell suspensions to IFNγ ELISPOT assays and the left over cells were frozen in 90% FCS and 10% DMSO in liquid nitrogen for storage. Peritoneal lavages contained 2 to 20×10⁶ cells that were used for flow cytometry analysis or frozen in liquid nitrogen by the same process used for spleens and bone marrow. Cell-free peritoneal lavage supernatants were stored at −20° C. Tumors were fixed in formalin for paraffin embedding and IHC to be conducted at a later time during the award.

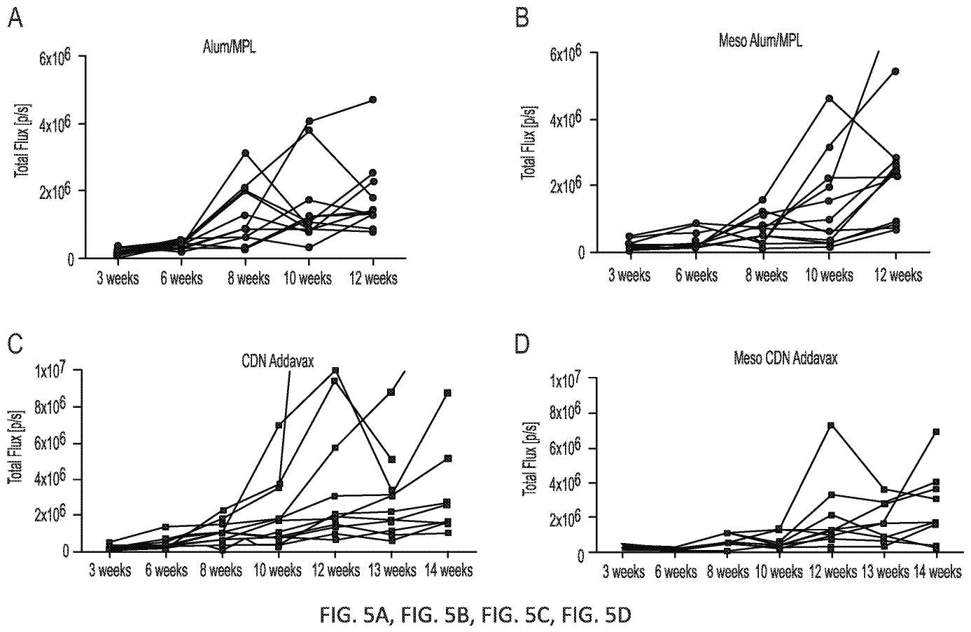

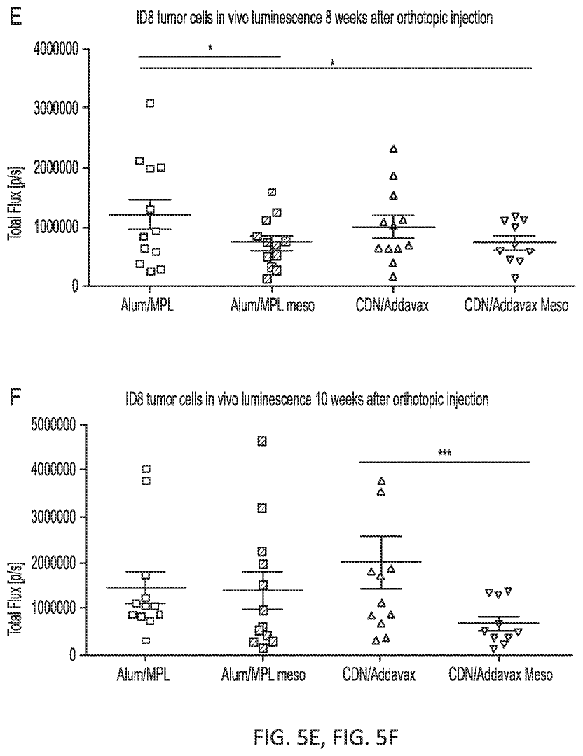

FIG. 5 shows representative examples of BLI per mouse and per week for tumor development (FIG. 5A) versus vaccine protection for group 2 (#2.43, upper panels FIG. 5B) and group 4 (#4.26 and 4.8, middle and lower panels FIG. 9B)

FIG. 7 shows the analysis of the lymphocytes in peritoneal lavages. Mice immunized with mesothelin/CDN/Addavax™ had significant increase of B and T cells. The characterization of CD4 T cells in naïve T cells (CD25−FoxP3−INFγ−), Treg (CD25+FoxP3+) or Th1(INFγ+) did not reveal any significant changes between groups even though a trend showing less Treg in groups 2 and 4 was perceptible. Significantly less memory cells (CD44+CD62L+) were present in mice of group 4. No changes in the percentages of CTL (CD8 IFNγ+) and CD8 PD1+ cells were measured either. These data support activation of adaptive immunity, both cellular (T cells) and humoral (B cells) after immunization with mesothelin/CDN/Addavax™.

FIG. 8 shows the analysis of myeloid cells in peritoneal lavages. A trend shows an increase of the percentage of myeloid cells in groups 2 and 4 compared with groups 1 and 3, respectively, but the difference did not reach significance (FIG. 8A). However and importantly, the composition of the myeloid compartment was significantly different in groups 1 and 2 versus groups 3 and 4. Groups immunized with the CDN/AddaVax™ combination presented more pro-inflammatory macrophages (M1, iNOS+), less myeloid derived suppressor cells (MDSC, Gr1+ CD11b+) and less PD-L1 expresser macrophages (FIG. 12B), supporting the potent activation of innate immunity by CDN/AddaVax™ combination towards tumor rejection.

Next, the percentages of live cells were analyzed that were leukocytes (CD45+ EpCam−) or likely tumor cells (CD45−) in the peritoneal lavages (FIG. 9). Significantly fewer leukocytes were present in the peritoneal lavages of group 4 to compare with the groups immunized with Alum/MPL, which was consistent with clinical observations. Strikingly, the characterization of tumor cells demonstrated a great heterogeneity between groups, indicating that ID8 cells were edited in function of the immunization types. FIG. 9B shows that EpCAM was profoundly down-regulated on CD45− cells from groups 1 and 2, consistent with extensive editing ID8-luc cells. In addition, PD-L1 was overexpressed by CD45− EpCAM+ cells in mice of group 4 to compare with those of group 3, indicating a higher stress of the tumor cells in group 4.

d. Cellular and Humoral Immune Response

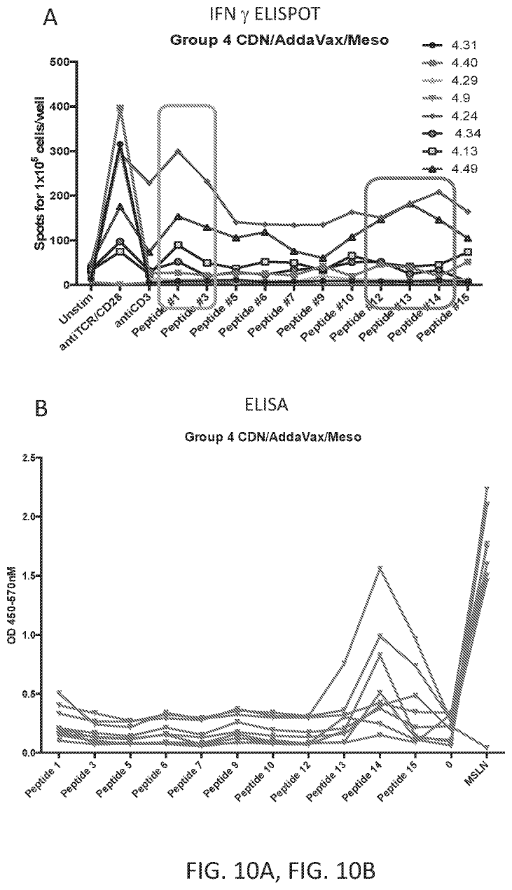

IFNγ Elispots were performed with splenocytes from immunized, tumor bearing mice. Splenocytes from animals immunized with mesothelin CDN and AddaVax™ produced IFNγ in presence of mesothelin peptides, particularly with peptides mapping in N- and C-terminal domains of mesothelin (FIG. 10A). ELISA assays were performed with sera from immunized, tumor bearing mice. Sera from animals immunized with mesothelin CDN and AddaVax™ contained IgG that bound to peptides specifically mapping the C-terminal domain of mesothelin (FIG. 10B).

ii. CONCLUSION

The evaluation of the ID-8 luc ovarian cancer model immunized with 10 μg of mesothelin in CDN 15 μg+Addavax™ appeared to significantly delay the development of metastatic ovarian cancer. Furthermore, analysis of the tumor and immune cells from mice vaccinated by the different adjuvants with and without mesothelin are revealing key distinctions in cellular immune responses triggered by CDN and Addavax™ against mesothelin.

Those skilled in the art will recognize, or be able to ascertain using no more than routine experimentation, many equivalents to the specific embodiments of the method and compositions described herein. Such equivalents are intended to be encompassed by the following claims.

D. Mesothelin-Based Vaccine Alone or Combined with Checkpoint Blockade

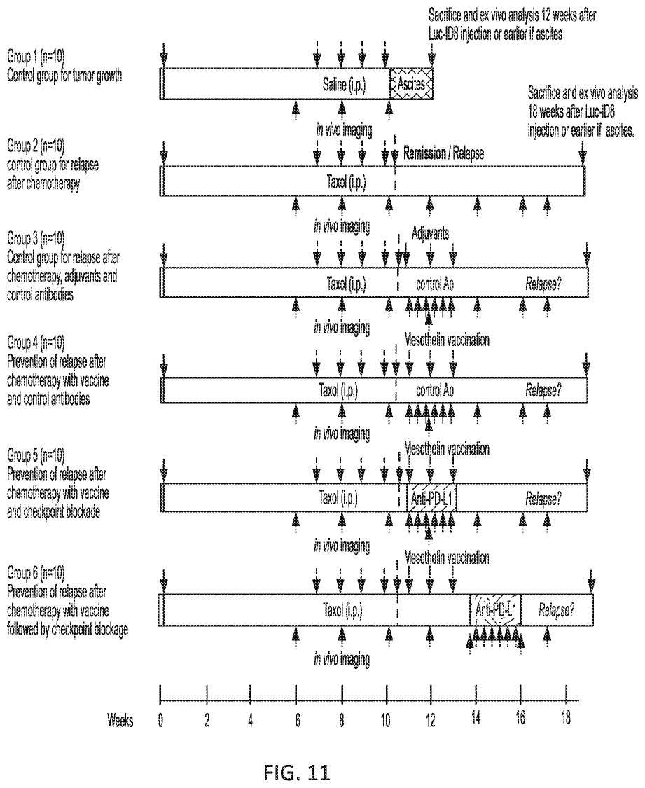

New strategies of prophylactic vaccination have been designed against ovarian cancer based on mesothelin combined with various adjuvants. Immunization of wild-type mice with human mesothelin protein combined with synthetic cyclic dinucleotides that activate type I IFN signaling (CDN) and depot adjuvant (AddaVax™) significantly impaired the growth of luciferase-transduced, mesothelin-expressing mouse ovarian tumor cells (Luc-ID8) injected in the ovary. These results indicate that the activation of type 1 IFN during immunization could modulate tumor invasive potential and immunogenicity. The activation of type 1 IFN signaling during immunization can improve prognosis and cancer therapeutic response to checkpoint blockade. The administration of mesothelin-based vaccine with type 1 IFN adjuvant during tumor remission phase can prevent or delay ovarian cancer relapse. These principles can be tested in wild-type mice orthotopically injected with Luc-ID8 cells. Tumor-bearing mice can be treated with Taxol and, after tumor regression, immunized with mesothelin combined with CDN/AddaVax™, with or without injections of anti-PD-L1 antibody. Tumor development and/or relapse can be followed by in vivo imaging (FIG. 11).

1. Background

Most patients with high-grade serous ovarian cancer are diagnosed with late stage disease, when survival rates remain poor and the relapse rates are high despite recent advances in surgical and pharmaceutical therapies. Initial or first-line chemotherapy fails to produce a remission in more than 70% of patients with ovarian cancer. In addition, approximately 40-50% of the women who achieve a remission after first-line chemotherapy will experience a recurrence of cancer within three years. The poor outcomes in ovarian cancer provide an impetus for the development of preventative agents and treatments to reduce mortality and suffering. The critical role of immune surveillance in ovarian cancer has been demonstrated by correlation of survival with tumor-infiltrating lymphocytes. Ovarian cancer is defined as an immunogenic tumor that exhibits a spontaneous antitumor immune response. The ability of endogenous T cells to destroy cancer cells can be improved by immunotherapy. The therapeutic efficacy of checkpoint blockade that releases tumor-infiltrating CTL inhibition via blocking of the CTLA-4 or the PD-1/PD-L1 pathways has been demonstrated in a variety of human malignancies. However, more than 70% of patients do not benefit from the current immunotherapeutic approaches.

2. Preclinical Study

A preclinical study to assess whether mesothelin-based vaccine adjuvanted with CDN can prevent ovarian cancer relapse, alone or combined with checkpoint blockade can be performed. Wild type C57-B16 mice were orthotopically injected with mesothelin-expressing Luc-ID8 mouse ovarian cancer cells. Tumor-bearing mice will be treated with Taxol and, after tumor regression, immunized with mesothelin combined with 2 types of adjuvants+/−injections of anti-PD-L1 antibody (FIG. 11).

FIG. 11 shows an experimental design of a mesothelin-based vaccine adjuvanted with CDN administered alone or in combination with a checkpoint blockade. C57Bl/6 mice were injected intraovary with Luc-ID8 cells and 6 weeks later were randomized by tumor size in 6 groups of 10 mice. Groups #2 and #6 are treated with Taxol (15 mg/kg×4, IP weekly) from weeks 7 to 10 after tumor injection.

Once in remission phase, mice from groups #4-#6 will receive mesothelin vaccine combined to 200 micrograms IP of anti-PD-L1 mAb (clone 10F.9G2, BioXcell) every 3 days, 6 times (group #5-6). Anti-PD-L1 antibodies will be administered during immunizations (group #5) or one week after the last immunization (group #6). Control antibodies will be administered during immunizations (group #4).

Control groups (groups #1-3) will receive PBS only (group #1), Taxol only (group #2), or Taxol and vaccine adjuvants with control antibody (group #3).

Tumor growth and ascites development are monitored by biweekly in vivo imaging and weighting, until more than 50% of mice in a group develop ascites, or for 18 weeks if no ascites develop.

### REFERENCES

1- Steven L Jacques. Optical properties of biological tissues: a review (2013) Phys. Med. Biol. 58 R37
2- Baert et al., The dark side of ID8-Luc2: pitfalls for luciferase tagged murine models for ovarian cancer. Journal for ImmunoTherapy of Cancer (2015) 3:57, DOI 10.1186/s40425-015-0102-0
3- Zhang, L., et al., Intratumoral T cells, recurrence, and survival in epithelial ovarian cancer. N Engl J Med, 2003. 348(3): p. 203-13.
4. Kandalaft, L. E., et al., Tumor immune surveillance and ovarian cancer: lessons on immune mediated tumor rejection or tolerance. Cancer Metastasis Rev, 2011. 30(1): p. 141-51.
5. De Felice, F., et al., Immunotherapy of Ovarian Cancer: The Role of Checkpoint Inhibitors. J Immunol Res, 2015. 2015: p. 191832.
6. Topalian, S. L., C. G. Drake, and D. M. Pardoll, Immune checkpoint blockade: a common denominator approach to cancer therapy. Cancer Cell, 2015. 27(4): p. 450-61.

We claim:

1. A composition comprising a mesothelin protein and an adjuvant.

2. The composition of claim 1, wherein the adjuvant is capable of binding to the stimulator of interferon genes (STING).

3. The composition of claim 1, wherein the adjuvant is cyclic dinucleotides (CDNs).

4. The composition of claim 2, wherein the CDNs are synthetic.

5. The composition of any one of claims 2-4, wherein the CDN is 2′3′-cGAMP.

6. The composition of any one of claims 1-5, wherein the mesothelin protein is recombinant.

7. The composition of any one of claims 1-6, wherein the mesothelin protein is full length human mesothelin protein.

8. The composition of any one of claims 1-7, further comprising a second adjuvant.

9. The composition of claim 8, wherein the second adjuvant is a squalene-based-oil-in-water emulsion

10. The composition of any one of claims 1-9, further comprising an immunomodulatory agent.

11. The composition of claim 10, wherein the immunomodulatory agent enhances the immune response.

12. The composition of any one of claims 10-11, wherein the immunomodulatory agent inhibits PD-1 or anti PD-L1

13. A method of treating cancer comprising administering to a subject a vaccine, wherein the vaccine comprises the composition of one of claims 1-12.

14. The method of claim 13, wherein the cancer is ovarian, lung or pancreatic cancer.

15. The method of claim 13, further comprising administering an immunomodulatory agent.

16. The method of claim 14, wherein the immunomodulatory agent enhances the immune response.

17. The method of any one of claims 14-16, wherein the immunomodulatory agent inhibits PD-1 or anti PD-L1.

18. The method of any one of claims 14-17, wherein the immunomodulatory agent is administered simultaneously with the vaccine.

19. The method of any one of claims 13-18, further comprising detecting an overexpression of mesothelin in the ovaries of the subject prior to administering the vaccine.

20. A method of triggering an immune response against mesothelin in a subject comprising administering to the subject the composition of any one of claims 1-12.

21. The method of claim 20, wherein the immune response is a Th1 immune response.

22. The method of claim 20, wherein the immune response is a Th2 immune response.

23. The method of claim 20, wherein the immune response is both a Th1 and Th2 immune response.

24. A method of immunizing a subject against ovarian cancer comprising administering to a subject a vaccine, wherein the vaccine comprises the composition of any one of claims 1-12.

25. The method of claim 24, wherein mesothelin specific antibodies are increased in the subject.

26. The method of any one of claims 24-25, wherein mesothelin-specific cytotoxic CD8+ T cells are elevated.

27. A method of slowing disease progression in a subject comprising administering to the subject the composition of any one of claims 1-12.

28. A method of reducing tumor burden in a subject comprising administering to the subject the composition of any one of claims 1-12.
